(19) United States
(12) Patent Application Publication
(10) Pub. No.: US 2012/0330197 A1
(43) Pub. Date: Dec. 27, 2012

(54) NONINVASIVE TREATMENT OF BLOOD VESSELS

(75) Inventors: Inder Raj S. Makin, Mesa, AZ (US); Michael H. Slayton, Tempe, AZ (US); Peter G. Barthe, Phoenix, AZ (US)

(73) Assignee: GUIDED THERAPY SYSTEMS, LLC, Mesa, AZ (US)

(21) Appl. No.: 13/601,742

(22) Filed: Aug. 31, 2012

Related U.S. Application Data
(63) Continuation of application No. 11/163,176, filed on Oct. 7, 2005, now abandoned.
(60) Provisional application No. 60/617,294, filed on Oct. 7, 2004.

(51) Int. Cl. A61N 7/02

(52) U.S. Cl. 601/3

(57) ABSTRACT

A non-invasive method and system for using ultrasound energy for the treatment of conditions resulting from vascular disorders is provided. In one embodiment, an image-treatment approach can be used to locate the blood vessel to be treated and then to ablate it non-invasively, while also monitoring the progress of the treatment. In another embodiment, a transducer is configured to deliver ultrasound energy to the regions of the superficial tissue (e.g., skin) such that the energy is deposited at the particular depth at which the vascular malformations are located below the skin surface. The ultrasound transducer can be driven at a number of different frequency regimes such that the depth and shape of energy concentration can match the region of treatment.

### CROSS-REFERENCE TO RELATED APPLICATIONS

This application is a continuation of U.S. application Ser. No. 11/163,176 on Oct. 7, 2005, which claims the benefit of priority to U.S. Provisional Application No. 60/617,294, filed on Oct. 7, 2004, each of which is incorporated in its entirety by reference herein.

### BACKGROUND

1. Field of the Invention

The present invention relates to an ultrasound therapy methods and systems, and in particular to a method and system for ultrasound treatment for superficial and peripheral blood vessels.

2. Description of the Related Art

Varicose veins (telangiectasia) are the clinical manifestation of underlying venous insufficiency. The venous insufficiency especially in the leg veins allows the venous blood to flow in the retrograde direction in the congested leg veins. The veins eventually dilate due to the increased venous pressure. The aberrant venous flow results in the leg veins from failure of the valves normally present in the veins, as well as the reduced muscle tone of the leg muscles. Further, varicosities of the leg veins result from chronically elevated venous pressure. Venous insufficiency can be present in the superficial or the deep veins, each pathology having its own set of sequelae. Varicose and spider veins are more prevalent in the female population.

Sclerotherapy, laser and intense-pulsed-light therapy, radio-frequency ablation, and surgical extirpation are the modern techniques used to ablate varicosities. During sclerotherapy a sclerosing agent (e.g., polidocanol, hypertonic sodium chloride, etc.) is injected in the dilated vein. A high degree of skill is required for this procedure. The treatment is ineffective in cases where a deeper aberrant vein is missed. Further, the technique has significant morbidity in cases where the agent extravasates outside the blood vessel. Transcutaneous laser or intense pulse light (IPL) are relevant only for small vascular malformations (such as) in the face. However, endovenous laser therapy, whereby a bare fiber is inserted in the varicose vein segment of the vein to coagulate and seal the vein, has proven to be quite effective for veins that are not very deep. The RF-energy-based catheters ablate the vein in a manner similar to the laser devices in coagulating the diseased blood vessel segment. Surgical techniques such as saphenectomy are sometimes used to ligate the dilated part of the veins but can be costly and may cause many complications.

Proliferate disease of the capillary tissue in the facial region also causes hemangionmas and port wine stain defects. These conditions are usually treated with lasers. However, the laser treatments can result in scarring, hyper/hypo pigmentation and other problems after treatment. Thus, more effective and non-invasive methods and systems for treating blood vessel disorders are needed.

### SUMMARY OF THE INVENTION

The present invention describes a non-invasive method and system for using ultrasound energy for the treatment of conditions resulting from vascular disorders, such as, for example, in the peripheral extremities and face. Ultrasound energy can be used for treatment of spider veins/engorged veins that are several millimeters in diameter and a up to 70 mm deep, as well to treat other vascular defects in the face and body. In one exemplary embodiment, an image-treatment approach can be used to locate the blood vessel to be treated and then to ablate it non-invasively, while also monitoring the progress of the treatment.

In another embodiment, an ultrasound system and method comprises a transducer and system configured to deliver ultrasound energy to the regions of the superficial tissue (e.g., skin) such that the energy can be deposited at the particular depth at which the vascular malformations (such as but not limited to varicose veins) are located below the skin surface. The ultrasound transducer can be driven at a number of different frequency regimes such that the depth and shape of energy concentration can match the region of treatment. The beam radiated from the transducer can be highly focused, weakly focused, and/or divergent, each in a cylindrical and/or spherical geometric configuration. The ultrasound source can also be planar to radiate a directive beam through the tissue. Further, the ultrasound field can be varied spatially and temporally by moving the source with respect to the tissue as well as pulsing the source in a pre-determined manner to achieve the optimal tissue effect on the sub-surficial vascular tissue.

### BRIEF DESCRIPTION OF THE DRAWINGS

The subject matter of the invention is particularly pointed out in the concluding portion of the specification. The invention, however, both as to organization and method of operation, may best be understood by reference to the following description taken in conjunction with the accompanying drawing figures, in which like parts may be referred to by like numerals:

FIG. 1 illustrates a block diagram of an exemplary ultrasound treatment system for treatment of blood vessel disorders in accordance with an exemplary embodiment of the present invention;

FIG. 2 illustrates a cross sectional diagram of an exemplary probe system in accordance with exemplary embodiments of the present invention;

FIGS. 3A and 3B illustrate block diagrams of an exemplary control system in accordance with exemplary embodiments of the present invention;

FIGS. 4A and 4B illustrate block diagrams of an exemplary probe system in accordance with exemplary embodiments of the present invention;

FIG. 5 illustrates a cross-sectional diagram of an exemplary transducer in accordance with an exemplary embodiment of the present invention;

FIGS. 6A and 6B illustrate cross-sectional diagrams of an exemplary transducer in accordance with exemplary embodiments of the present invention;

FIG. 7 illustrates exemplary transducer configurations for ultrasound treatment in accordance with various exemplary embodiments of the present invention;

FIGS. 8A and 8B illustrate cross-sectional diagrams of an exemplary transducer in accordance with another exemplary embodiment of the present invention;

FIG. 9 illustrates an exemplary transducer configured as a two-dimensional array for ultrasound treatment in accordance with an exemplary embodiment of the present invention;

FIGS. 10A-10F illustrate cross-sectional diagrams of exemplary transducers in accordance with other exemplary embodiments of the present invention;

FIG. 11 illustrates a schematic diagram of an acoustic coupling and cooling system in accordance with an exemplary embodiment of the present invention; and

FIG. 12 illustrates a block diagram of a treatment system comprising an ultrasound treatment subsystem combined with additional subsystems and methods of treatment monitoring and/or treatment imaging as well as a secondary treatment subsystem in accordance with an exemplary embodiment of the present invention.

### DETAILED DESCRIPTION

The present invention may be described herein in terms of various functional components and processing steps. It should be appreciated that such components and steps may be realized by any number of hardware components configured to perform the specified functions. For example, the present invention may employ various medical treatment devices, visual imaging and display devices, input terminals and the like, which may carry out a variety of functions under the control of one or more control systems or other control devices. In addition, the present invention may be practiced in any number of medical or treatment contexts and that the exemplary embodiments relating to a method and system for treatment of blood vessel disorders as described herein are merely indicative of the exemplary applications for the invention. For example, the principles, features and methods discussed may be applied to any medical or other tissue or treatment application. Further, various aspects of the present invention may be suitably applied to other applications

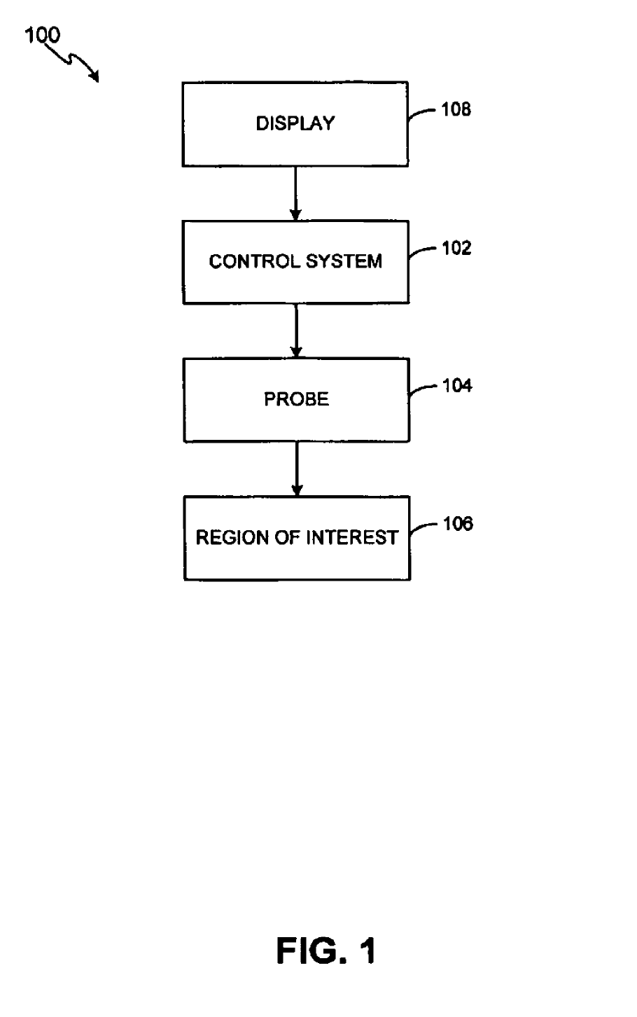

In accordance with various aspects of the present invention, a non-invasive method and system for the treatment of peripheral vascular defects is described. An ultrasound transducer and system is configured to deliver ultrasound energy to the user specified depth and zone where the vascular defects are to be treated. For example, with the reference to an exemplary block diagram illustrated in FIG. 1, exemplary blood vessel disorder treatment system 100 comprises an exemplary transducer system 102 that can be coupled to control system 104 and/or display 106 to provide ultrasound therapy, imaging and/or temperature or other tissue parameter monitoring to one or more region of interest (ROI) 110. The ultrasound beam can be spatially and/or temporally modified to match the adequate treatment of the aberrant vessels in the treatment zone.

For example, in one embodiment, blood vessel disorder treatment system 100 is configured with the ability to provide non-invasive methods and systems for using ultrasound energy for the treatment of conditions resulting from vascular disorders, such as, for example, in the peripheral extremities and face. As used herein, the phrases “blood vessel disorders”, “vascular disorders” and the like include, but are not limited to peripheral vascular deformities such as, for example, varicose veins, spider veins, deep vein disorders, facial hemangiomas or port wine stains, and/or the like.

In accordance with an exemplary embodiment, control system 104 and transducer system 102 can be suitably configured to deliver conformal ultrasound therapeutic energy to ROI 110 for treatment of spider veins/engorged veins that are several millimeters in diameter and a up to 70 mm deep, as well to treat other vascular defects in the face and body.

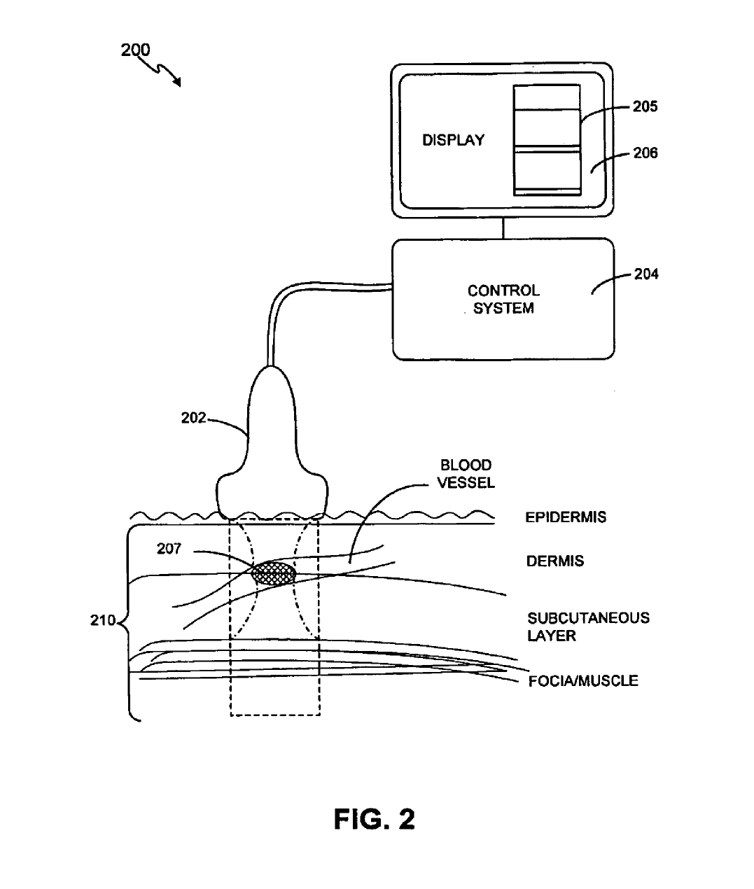

Exemplary systems for treatment can facilitate the combination of imaging (targeting and monitoring) mechanisms with the therapy mechanisms configured with a single energy modality. Due to its non-invasive nature, these treatment systems and methods can enable the management of a disease over repeat procedures until the clinical condition shows improvement. An exemplary ultrasound therapy system of FIG. 1 is further illustrated in an exemplary embodiment in FIG. 2. A therapy transducer system 200 includes a transducer/probe 202 connected to a control system 204, and display 206, in combination may provide therapy, imaging, and/or temperature or other tissue parameters monitoring to region of interest 210. Exemplary transducer system 200 is configured for first, imaging and display of region of interest 210 for localization of the treatment area and surrounding structures, second, delivery of focused, unfocused, or defocused ultrasound energy at a depth, distribution, timing, and energy level to achieve the desired therapeutic effect of thermal ablation to treat cellulite, and third to monitor the treatment area and surrounding structures before, during, and after therapy to plan and assess the results and/or provide feedback to control system 204 and/or an operator.

Exemplary transducer probe 202 can be configured to be suitably controlled and/or operated in various manners. For example, transducer probe 202 may be configured for use within an ultrasound treatment system, an ultrasound imaging system and/or an ultrasound imaging, therapy, and/or treatment monitoring system, including motion control subsystems.

Control system 204 can be configured with one or more subsystems, processors, input devices, displays and/or the like. Display 206 may be configured to image and/or monitor ROI 210 and/or any particular sub-region within ROI 210. Display 206 can be configured for two-dimensional, three-dimensional, real-time, analog, digital and/or any other type of imaging. Exemplary embodiments of both control system 204 and display 206 are described in greater detail herein.

In one embodiment, region of interest 210 can comprise any particular vessel or group of vessels and/or any portion within a vessel. Exemplary transducer system 200, is configured to provide cross-sectional two-dimensional imaging of the region 207, displayed as an image 205, with a controlled thermal lesion confined approximately to approximately 0.1 to 5 mm in diameter in order facilitate ablation of the vessel and approximately 3 to 20 mm in diameter in order facilitate ablation of the vessel. The lesion may be any shape to provide ablation of the blood vessel. For example, spherical, ellipsoid, and/or cigar shaped lesions may be effective for ablation purposes. Methods for treating blood vessels are disclosed further herein.

Transducer system 200 can be configured with the ability to controllably produce conformal treatment areas in superficial human tissue within region of interest 210 through precise spatial and temporal control of acoustic energy deposition. In accordance with an exemplary embodiment, control system 204 and transducer probe 202 can be suitably configured for spatial control of the acoustic energy by controlling the manner of distribution of the acoustical energy. For example, spatial control may be realized through selection of the type of one or more transducer configurations insonifying region of interest 210, selection of the placement and location of transducer probe 202 for delivery of acoustical energy relative to region-of-interest 210, e.g., transducer probe 202 configured for scanning over part or whole of region-of-interest 210 to deliver conformal ultrasound therapeutic energy to treat spider veins/engorged veins that are several millimeters in diameter and a up to 70 mm deep, as well to treat other vascular defects in the face and body.

In another embodiment, transducer system 200 comprises transducer probe 202 configured to deliver ultrasound energy to the regions of the superficial tissue (ROI 210) such that the energy can be deposited at the particular depth at which the vascular malformations (such as but not limited to varicose veins) are located below the skin surface. Transducer probe 202 can be driven at a number of different frequency regimes such that the depth and shape of energy concentration can match ROI 210. The beam radiated from transducer probe 202 can be highly focused, weakly focused, and/or divergent, each in a cylindrical and/or spherical geometric configuration. The ultrasound source can also be planar to radiate a directive beam through the tissue. Further, the ultrasound field can be varied spatially and temporally by moving the source with respect to the tissue as well as pulsing the source in a pre-determined manner to achieve the optimal tissue effect on the sub-surficial vascular tissue.

In another exemplary embodiment, and in the case of deep engorged veins, a catheter ablative technique may be extremely difficult and/or impossible. Accordingly, transducer system 200 can be configured to suitably control transducer probe 202 to operate in various manners. For example, transducer probe 202 may be configured for use within an ultrasound treatment system, an ultrasound imaging system and/or an ultrasound imaging, therapy, and/or treatment monitoring system, including motion control subsystems. These subsystems can help facilitate ablation of a specific occlusion within the blood vessels to facilitate treatment.

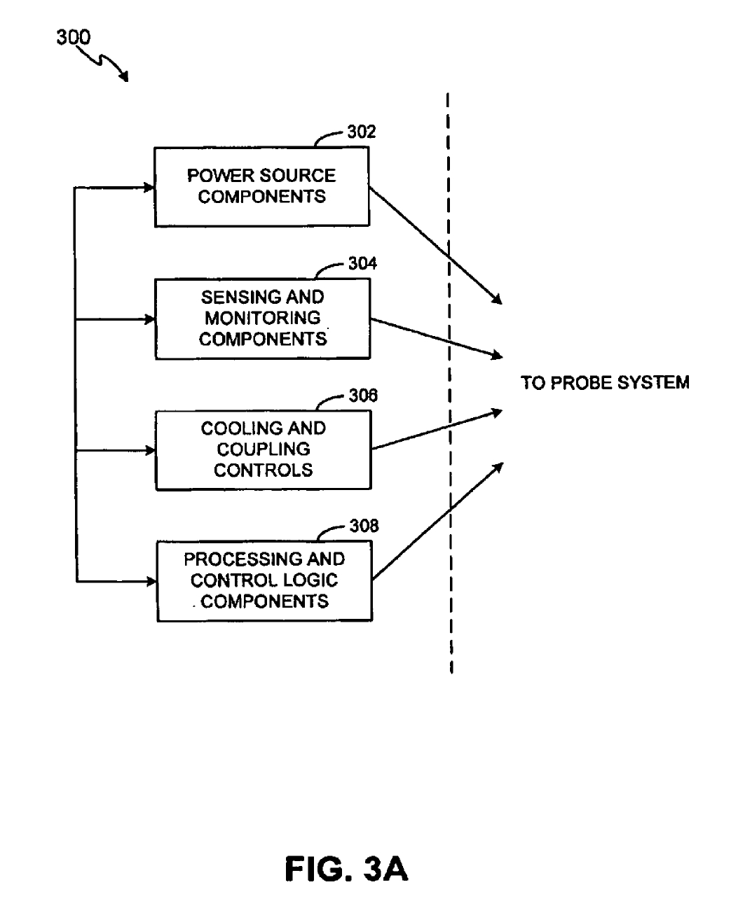

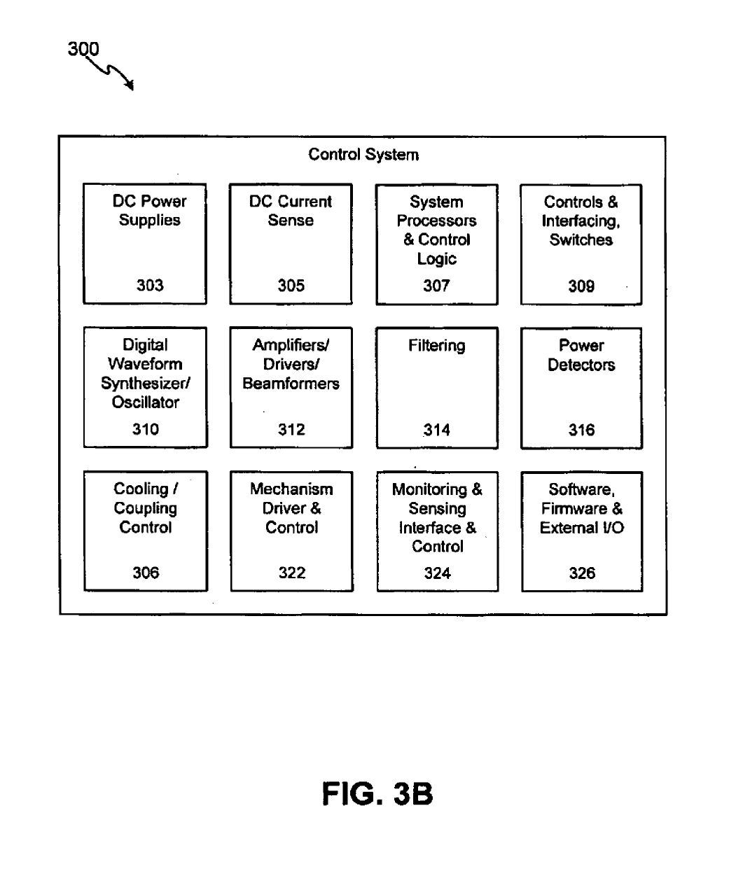

A As previously described, control systems 102 and 204 may be configured in various manners with various subsystems and subcomponents. With reference to FIGS. 3A and 3B, in accordance with exemplary embodiments, an exemplary control system 300 can be configured for coordination and control of the entire therapeutic treatment process in accordance with the adjustable settings made by a therapeutic treatment system user. For example, control system 300 can suitably comprise power source components 302, sensing and monitoring components 304, cooling and coupling controls 306, and/or processing and control logic components 308. Control system 300 can be configured and optimized in a variety of ways with more or less subsystems and components to implement the therapeutic system for treatment of blood vessel disorders, and the embodiment in FIGS. 3A and 3B are merely for illustration purposes.

For example, for power sourcing components 302, control system 300 can comprise one or more direct current (DC) power supplies 303 configured to provide electrical energy for entire control system 300, including power required by a transducer electronic amplifier/driver 312. A DC current sense device 305 can also be provided to confirm the level of power going into amplifiers/drivers 312 for safety and monitoring purposes.

Amplifiers/drivers 312 can comprise multi-channel or single channel power amplifiers and/or drivers. In accordance with an exemplary embodiment for transducer array configurations, amplifiers/drivers 312 can also be configured with a beamformer to facilitate array focusing. An exemplary beamformer can be electrically excited by an oscillator/digitally controlled waveform synthesizer 310 with related switching logic.

The power sourcing components can also include various filtering configurations 314. For example, switchable harmonic filters and/or matching may be used at the output of amplifier/driver 312 to increase the drive efficiency and effectiveness. Power detection components 316 may also be included to confirm appropriate operation and calibration. For example, electric power and other energy detection components 316 may be used to monitor the amount of power going to an exemplary probe system.

Various sensing and monitoring components 304 may also be suitably implemented within control system 300. For example, in accordance with an exemplary embodiment, monitoring, sensing and interface control components 324 may be configured to operate with various motion detection systems implemented within transducer probe 104 to receive and process information such as acoustic or other spatial and temporal information from a region of interest. Sensing and monitoring components can also include various controls, interfacing and switches 309 and/or power detectors 316. Such sensing and monitoring components 304 can facilitate open-loop and/or closed-loop feedback systems within treatment system 100.

For example, in such an open-loop system, a system user can suitably monitor the imaging and or other spatial or temporal parameters and then adjust or modify same to accomplish a particular treatment objective. Instead of, or in combination with open-loop feedback configurations, an exemplary treatment system can comprise a closed-loop feedback system, wherein images and/or spatial/temporal parameters can be suitably monitored within monitoring component to generate signals.

During operation of exemplary treatment system 100, a lesion configuration of a selected size, shape, orientation is determined. Based on that lesion configuration, one or more spatial parameters are selected, along with suitable temporal parameters, the combination of which yields the desired conformal lesion. Operation of the transducer can then be initiated to provide the conformal lesion or lesions. Open and/or closed-loop feedback systems can also be implemented to monitor the spatial and/or temporal characteristics, and/or other tissue parameter monitoring, to further control the conformal lesions.

Cooling/coupling control systems 306 may be provided to remove waste heat from exemplary probe 104, provide a controlled temperature at the superficial tissue interface and deeper, for example into blood and/or tissue, and/or provide acoustic coupling from transducer probe 104 to region-of-interest 106. Such cooling/coupling control systems 306 can also be configured to operate in both open-loop and/or closed-loop feedback arrangements with various coupling and feedback components.

Processing and control logic components 308 can comprise various system processors and digital control logic 307, such as one or more of microcontrollers, microprocessors, field-programmable gate arrays (FPGAs), computer boards, and associated components, including firmware and control software 326, which interfaces to user controls and interfacing circuits as well as input/output circuits and systems for communications, displays, interfacing, storage, documentation, and other useful functions. System software and firmware 326 controls all initialization, timing, level setting, monitoring, safety monitoring, and all other system functions required to accomplish user-defined treatment objectives. Further, various control switches 308 can also be suitably configured to control operation.

An exemplary transducer probe 104 can also be configured in various manners and comprise a number of reusable and/or disposable components and parts in various embodiments to facilitate its operation. For example, transducer probe 104 can be configured within any type of transducer probe housing or arrangement for facilitating the coupling of transducer to a tissue interface, with such housing comprising various shapes, contours and configurations depending on the particular treatment application. For example, in accordance with an exemplary embodiment, transducer probe 104 can be depressed against a tissue interface whereby blood perfusion is partially or wholly cut-off, and tissue flattened in superficial treatment region-of-interest 106. Transducer probe 104 can comprise any type of matching, such as for example, electric matching, which may be electrically switchable; multiplexer circuits and/or aperture/element selection circuits; and/or probe identification devices, to certify probe handle, electric matching, transducer usage history and calibration, such as one or more serial EEPROM (memories). Transducer probe 104 may also comprise cables and connectors; motion mechanisms, motion sensors and encoders; thermal monitoring sensors; and/or user control and status related switches, and indicators such as LEDs. For example, a motion mechanism in probe 104 may be used to controllably create multiple lesions, or sensing of probe motion itself may be used to controllably create multiple lesions and/or stop creation of lesions, e.g. for safety reasons if probe 104 is suddenly jerked or is dropped. In addition, an external motion encoder arm may be used to hold the probe during use, whereby the spatial position and attitude of probe 104 is sent to the control system to help controllably create lesions. Furthermore, other sensing functionality such as profilometers or other imaging modalities may be integrated into the probe in accordance with various exemplary embodiments.

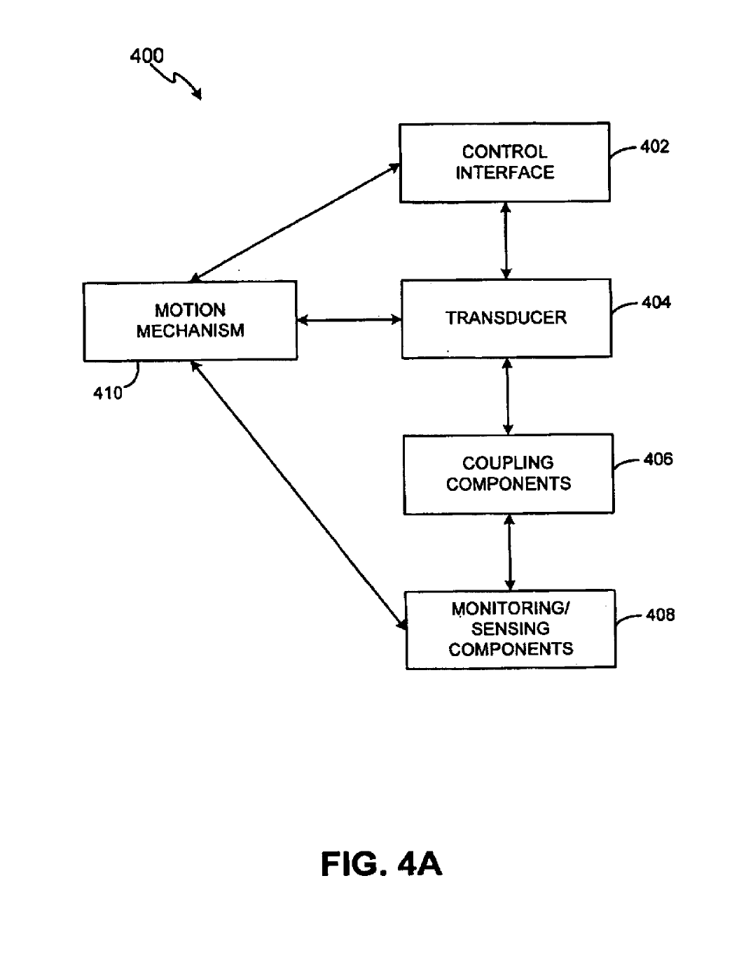

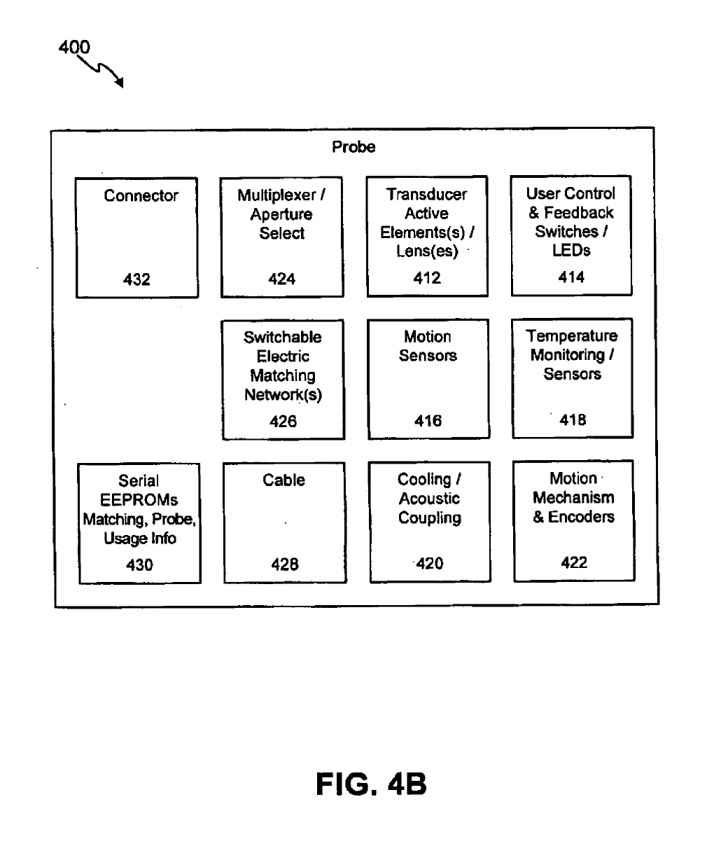

With reference to FIGS. 4A and 4B, in accordance with an exemplary embodiment, a transducer probe 400 can comprise a control interface 402, a transducer 404, coupling components 406, and monitoring/sensing components 408, and/or motion mechanism 410. However, transducer probe 400 can be configured and optimized in a variety of ways with more or less parts and components to provide ultrasound energy for treatment of blood vessel disorders, and the embodiment in FIGS. 4A and 4B are merely for illustration purposes.

In accordance with an exemplary embodiment of the present invention, transducer probe 400 is configured to deliver energy over varying temporal and/or spatial distributions in order to provide energy effects and initiate responses in a region of interest. These effects can include, for example, thermal, cavitational, hydrodynamic, and resonance induced tissue effects. For example, exemplary transducer probe 400 can be operated under one or more frequency ranges to provide two or more energy effects and initiate one or more responses in the region of interest. In addition, transducer probe 400 can also be configured to deliver planar, defocused and/or focused energy to a region of interest to provide two or more energy effects and to initiate one or more reactions. These responses can include, for example, diathermy, hemostasis, revascularization, angiogenesis, growth of interconnective tissue, tissue reformation, ablation of existing tissue, protein synthesis and/or enhanced cell permeability. These and various other exemplary embodiments for such combined ultrasound treatment, effects and responses are more fully set forth in U.S. patent application Ser. No. 10/950,112, entitled “Method and System for Combined Ultrasound Treatment,” Filed Sep. 24, 2004 and incorporated herein by reference.

Control interface 402 is configured for interfacing with control system 300 to facilitate control of transducer probe 400. Control interface components 402 can comprise multiplexer/aperture select 424, switchable electric matching networks 426, serial EEPROMs and/or other processing components and matching and probe usage information 430 and interface connectors 432.

Coupling components 406 can comprise various devices to facilitate coupling of transducer probe 400 to a region of interest. For example, coupling components 406 can comprise cooling and acoustic coupling system 420 configured for acoustic coupling of ultrasound energy and signals. Acoustic cooling/coupling system 420 with possible connections such as manifolds may be utilized to couple sound into the region-of-interest, control temperature at the interface and deeper, for example into blood and/or tissue, provide liquid-filled lens focusing, and/or to remove transducer waste heat. Coupling system 420 may facilitate such coupling through use of various coupling mediums, including air and other gases, water and other fluids, gels, solids, and/or any combination thereof, or any other medium that allows for signals to be transmitted between transducer active elements 412 and a region of interest. In addition to providing a coupling function, in accordance with an exemplary embodiment, coupling system 420 can also be configured for providing temperature control during the treatment application. For example, coupling system 420 can be configured for controlled cooling of an interface surface or region between transducer probe 400 and a region of interest and beyond and beyond by suitably controlling the temperature of the coupling medium. The suitable temperature for such coupling medium can be achieved in various manners, and utilize various feedback systems, such as thermocouples, thermistors or any other device or system configured for temperature measurement of a coupling medium. Such controlled cooling can be configured to further facilitate spatial and/or thermal energy control of transducer probe 400.

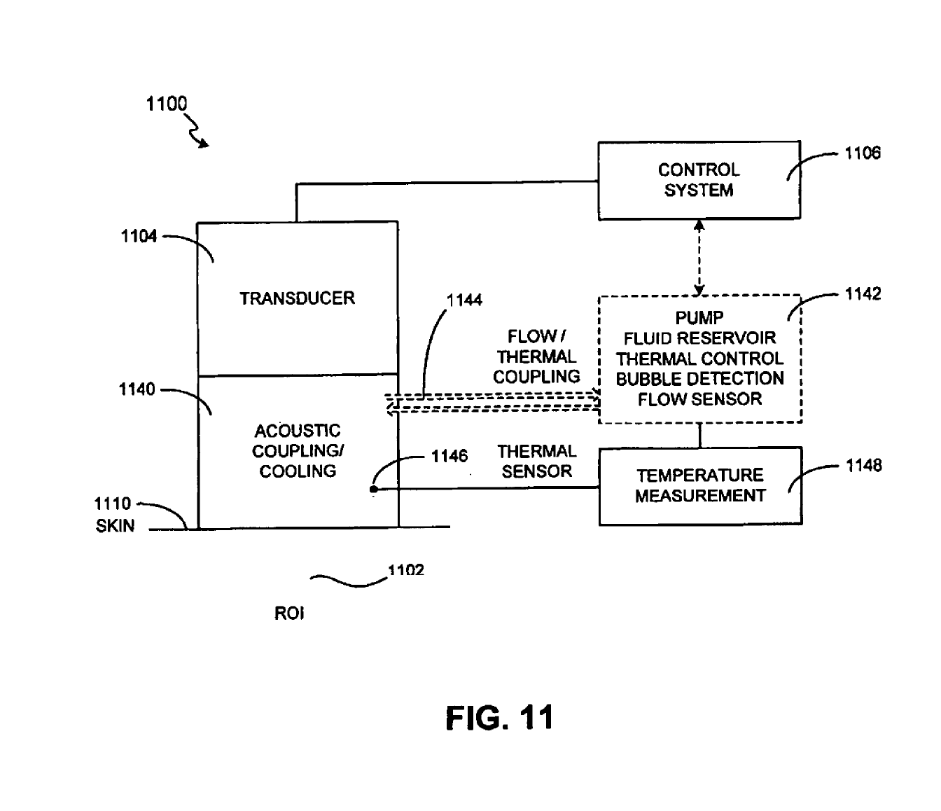

In accordance with an exemplary embodiment, with additional reference to FIG. 11, acoustic coupling and cooling 1140 can be provided to acoustically couple energy and imaging signals from transducer probe 1104 to and from the region of interest 1106, to provide thermal control at the probe to region-of-interest interface 1110 and deeper, for example into blood and/or tissue, and to remove potential waste heat from the transducer probe at region 1144. Temperature monitoring can be provided at the coupling interface via a thermal sensor 1146 to provide a mechanism of temperature measurement 1148 and control via control system 1102 and a thermal control system 1142. Thermal control may consist of passive cooling such as via heat sinks or natural conduction and convection or via active cooling such as with peltier thermoelectric coolers, refrigerants, or fluid-based systems comprised of pump, fluid reservoir, bubble detection, flow sensor, flow channels/tubing 1144 and thermal control 1142.

Monitoring and sensing components 408 can comprise various motion and/or position sensors 416, temperature monitoring sensors 418, user control and feedback switches 414 and other like components for facilitating control by control system 300, e.g., to facilitate spatial and/or temporal control through open-loop and closed-loop feedback arrangements that monitor various spatial and temporal characteristics.

Motion mechanism 410 can comprise manual operation, mechanical arrangements, or some combination thereof. For example, a motion mechanism 422 can be suitably controlled by control system 300, such as through the use of accelerometers, encoders or other position/orientation devices 416 to determine and enable movement and positions of transducer probe 400. Linear, rotational or variable movement can be facilitated, e.g., those depending on the treatment application and tissue contour surface.

Transducer 404 can comprise one or more transducers configured for producing conformal lesions of thermal injury in superficial human tissue within a region of interest through precise spatial and temporal control of acoustic energy deposition. Transducer 404 can also comprise one or more transduction elements and/or lenses 412. The transduction elements can comprise a piezoelectrically active material, such as lead zirconante titanate (PZT), or any other piezoelectrically active material, such as a piezoelectric ceramic, crystal, plastic, and/or composite materials, as well as lithium niobate, lead titanate, barium titanate, and/or lead metaniobate. In addition to, or instead of, a piezoelectrically active material, transducer 404 can comprise any other materials configured for generating radiation and/or acoustical energy. Transducer 404 can also comprise one or more matching layers configured along with the transduction element such as coupled to the piezoelectrically active material. Acoustic matching layers and/or damping may be employed as necessary to achieve the desired electroacoustic response.

In accordance with an exemplary embodiment, the thickness of the transduction element of transducer 404 can be configured to be uniform. That is, a transduction element 412 can be configured to have a thickness that is substantially the same throughout. In accordance with another exemplary embodiment, the thickness of a transduction element 412 can also be configured to be variable. For example, transduction element(s) 412 of transducer 404 can be configured to have a first thickness selected to provide a center operating frequency of a lower range, for example from approximately 1 MHz to 5 MHz. Transduction element 404 can also be configured with a second thickness selected to provide a center operating frequency of a higher range, for example from approximately 5 MHz to 15 MHz or more. Transducer 404 can be configured as a single broadband transducer excited with at least two or more frequencies to provide an adequate output for generating a desired response. Transducer 404 can also be configured as two or more individual transducers, wherein each transducer comprises one or more transduction element. The thickness of the transduction elements can be configured to provide center-operating frequencies in a desired treatment range. For example, transducer 404 can comprise a first transducer configured with a first transduction element having a thickness corresponding to a center frequency range of approximately 1 MHz to 5 MHz, and a second transducer configured with a second transduction element having a thickness corresponding to a center frequency of approximately 5 MHz to 15 MHz or more.

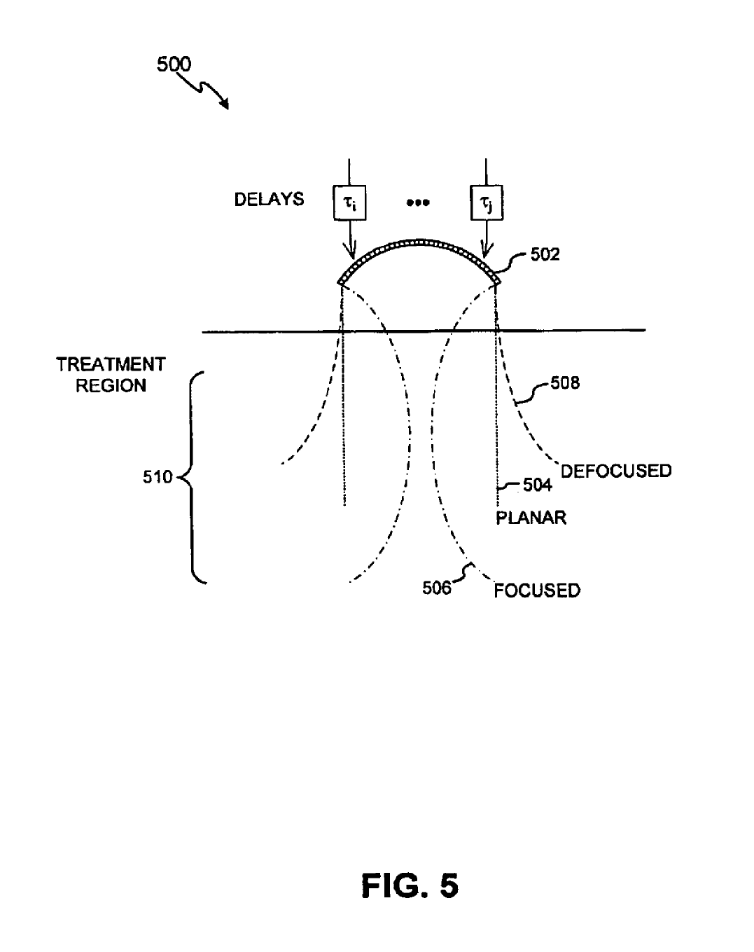

Transducer 404 may be composed of one or more individual transducers in any combination of focused, planar, or unfocused single-element, multi-element, or array transducers, including 1-D, 2-D, and annular arrays; linear, curvilinear, sector, or spherical arrays; spherically, cylindrically, and/or electronically focused, defocused, and/or lensed sources. For example, with reference to an exemplary embodiment depicted in FIG. 5, transducer 500 can be configured as an acoustic array to facilitate phase focusing. That is, transducer 500 can be configured as an array of electronic apertures that may be operated by a variety of phases via variable electronic time delays. By the term “operated,” the electronic apertures of transducer 500 may be manipulated, driven, used, and/or configured to produce and/or deliver an energy beam corresponding to the phase variation caused by the electronic time delay. For example, these phase variations can be used to deliver defocused beams, planar beams, and/or focused beams, each of which may be used in combination to achieve different physiological effects in a region of interest 510. Transducer 500 may additionally comprise any software and/or other hardware for generating, producing and or driving a phased aperture array with one or more electronic time delays.

Transducer 500 can also be configured to provide focused treatment to one or more regions of interest using various frequencies. In order to provide focused treatment, transducer 500 can be configured with one or more variable depth devices to facilitate treatment. For example, transducer 500 may be configured with variable depth devices disclosed in U.S. patent application Ser. No. 10/944,500, entitled “System and Method for Variable Depth Ultrasound”, filed on Sep. 16, 2004, having at least one common inventor and a common Assignee as the present application, and incorporated herein by reference. In addition, transducer 500 can also be configured to treat one or more additional ROI 510 through the enabling of sub-harmonics or pulse-echo imaging, as disclosed in U.S. patent application Ser. No. 10/944,499, entitled “Method and System for Ultrasound Treatment with a Multi-directional Transducer”, filed on Sep. 16, 2004, having at least one common inventor and a common Assignee as the present application, and also incorporated herein by reference.

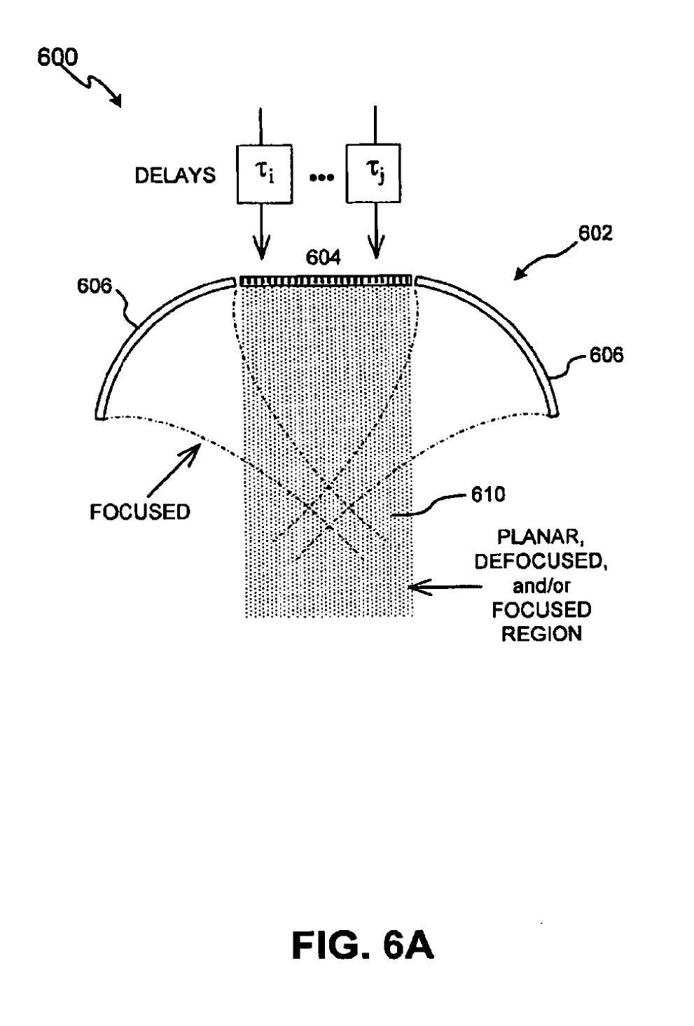

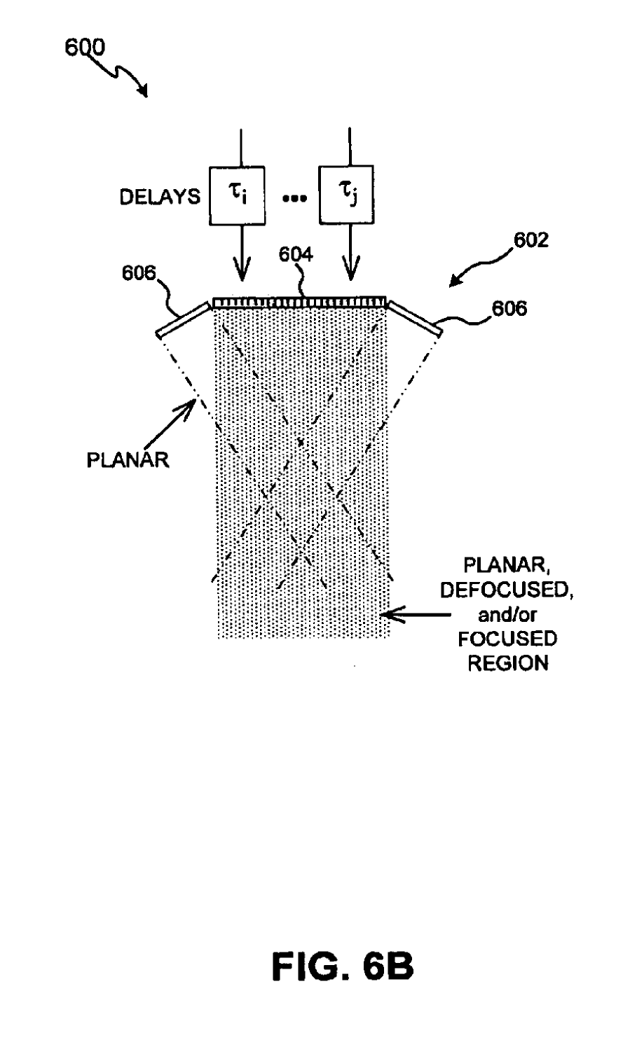

Moreover, any variety of mechanical lenses or variable focus lenses, e.g. liquid-filled lenses, may also be used to focus and or defocus the sound field. For example, with reference to exemplary embodiments depicted in FIGS. 6A and 6B, transducer 600 may also be configured with an electronic focusing array 604 in combination with one or more transduction elements 606 to facilitate increased flexibility in treating ROI 610. Array 604 may be configured in a manner similar to transducer 502. That is, array 604 can be configured as an array of electronic apertures that may be operated by a variety of phases via variable electronic time delays, for example, T1, T2 . . . Tj. By the term “operated,” the electronic apertures of array 604 may be manipulated, driven, used, and/or configured to produce and/or deliver energy in a manner corresponding to the phase variation caused by the electronic time delay. For example, these phase variations can be used to deliver defocused beams, planar beams, and/or focused beams, each of which may be used in combination to achieve different physiological effects in ROI 610.

Transduction elements 606 may be configured to be concave, convex, and/or planar. For example, in an exemplary embodiment depicted in FIG. 6A, transduction elements 606A are configured to be concave in order to provide focused energy for treatment of ROI 610. Additional embodiments are disclosed in U.S. patent application Ser. No. 10/944,500, entitled “Variable Depth Transducer System and Method”, and again incorporated herein by reference.

In another exemplary embodiment, depicted in FIG. 6B, transduction elements 606B can be configured to be substantially flat in order to provide substantially uniform energy to ROI 610. While FIGS. 6A and 6B depict exemplary embodiments with transduction elements 604 configured as concave and substantially flat, respectively, transduction elements 604 can be configured to be concave, convex, and/or substantially flat. In addition, transduction elements 604 can be configured to be any combination of concave, convex, and/or substantially flat structures. For example, a first transduction element can be configured to be concave, while a second transduction element can be configured to be substantially flat.

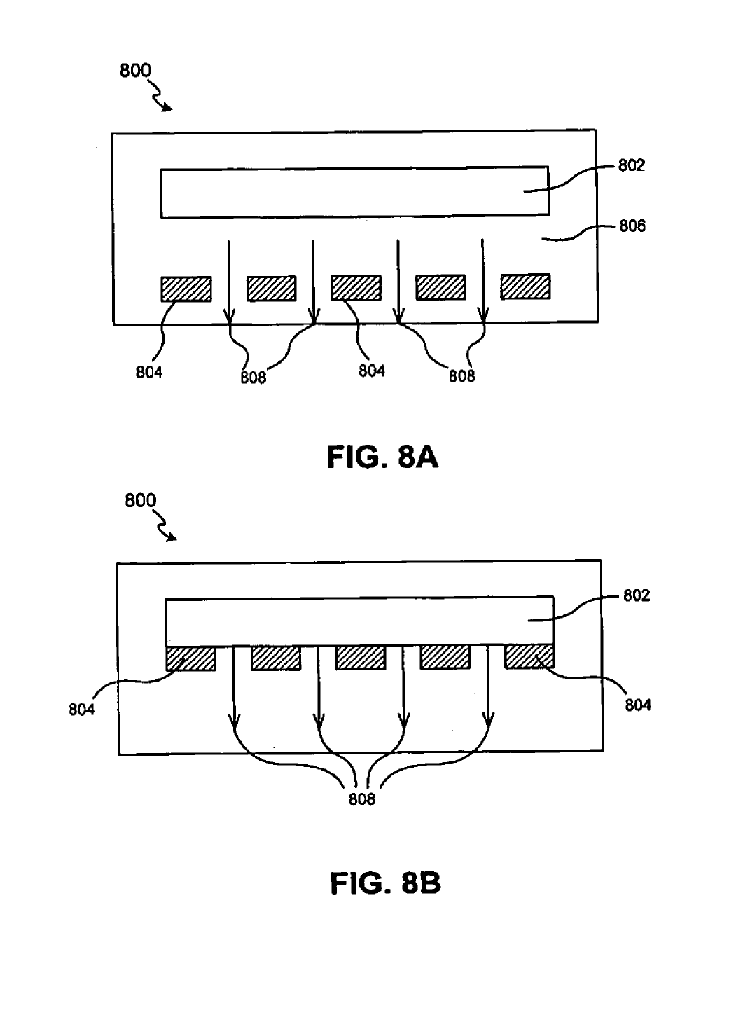

With reference to FIGS. 8A and 8B, transducer 404 can be configured as single-element arrays, wherein a single-element 802, e.g., a transduction element of various structures and materials, can be configured with a plurality of masks 804, such masks comprising ceramic, metal or any other material or structure for masking or altering energy distribution from element 802, creating an array of energy distributions 808. Masks 804 can be coupled directly to element 802 or separated by a standoff 806, such as any suitably solid or liquid material.

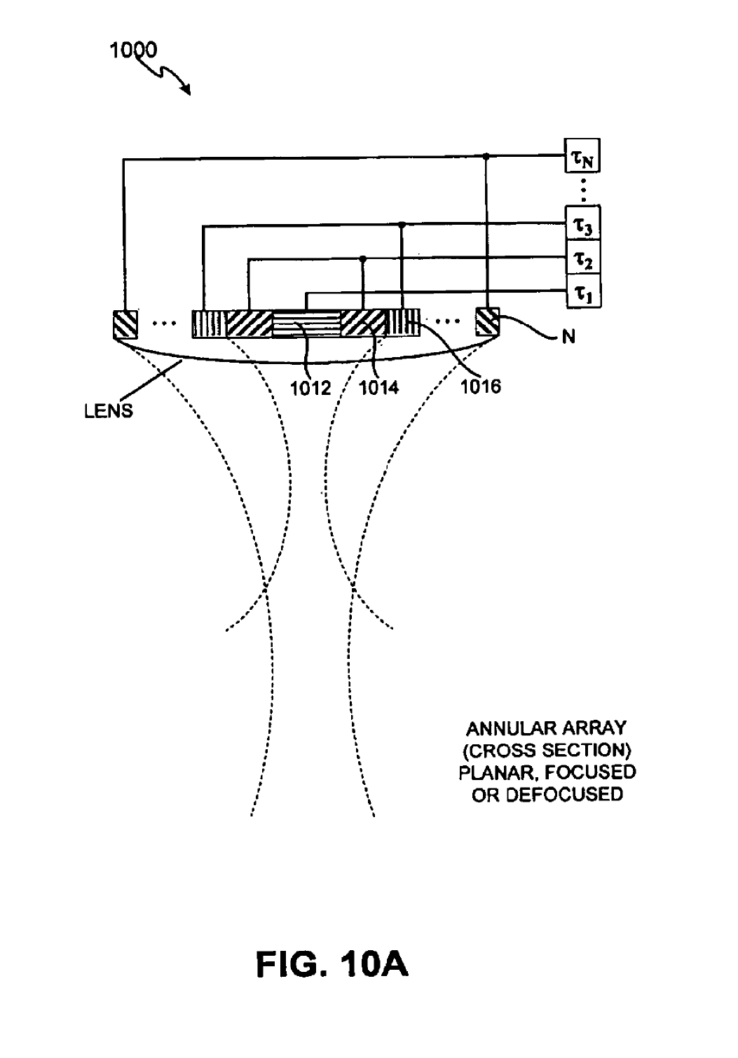

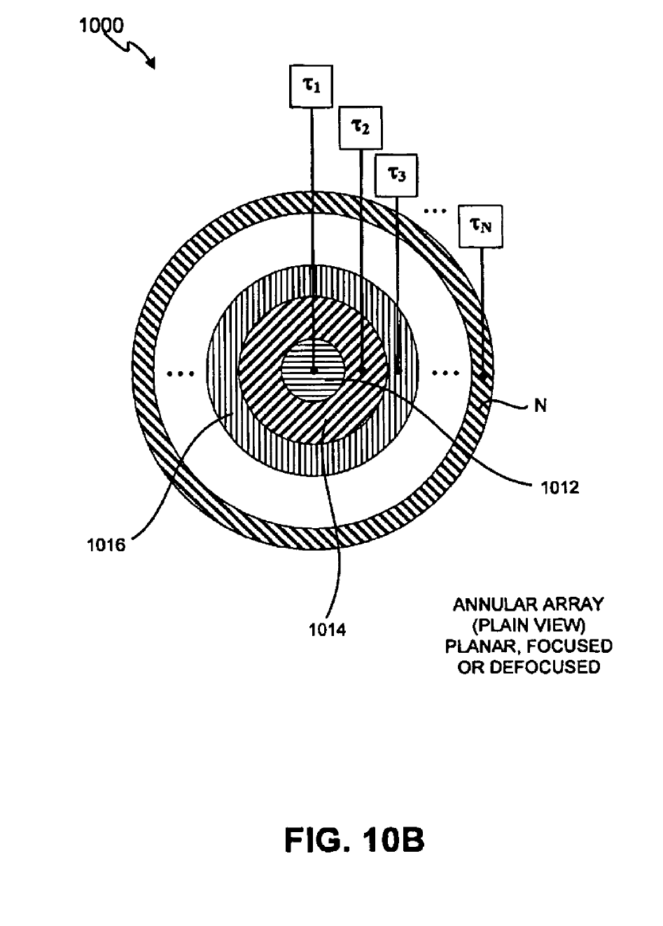

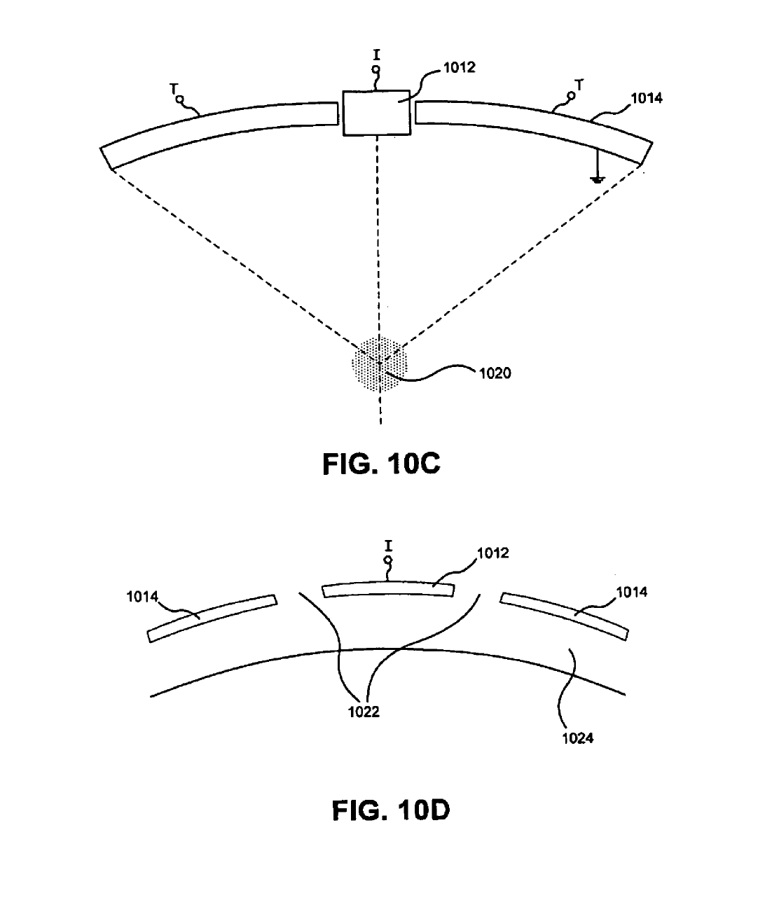

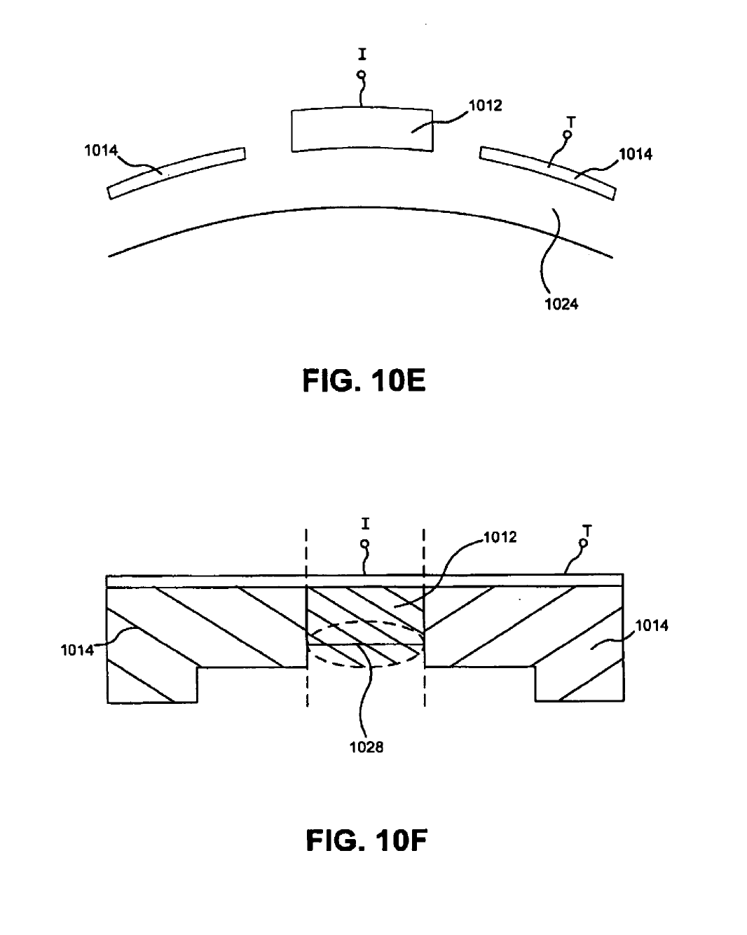

An exemplary transducer 404 can also be configured as an annular array to provide planar, focused and/or defocused acoustical energy. For example, with reference to FIGS. 10A and 10B, in accordance with an exemplary embodiment, an annular array 1000 can comprise a plurality of rings 1012, 1014, 1016 to N. Rings 1012, 1014, 1016 to N can be mechanically and electrically isolated into a set of individual elements, and can create planar, focused, or defocused waves. For example, such waves can be centered on-axis, such as by methods of adjusting corresponding transmit and/or receive delays, T1, T2, T3 . . . TN. An electronic focus can be suitably moved along various depth positions, and can enable variable strength or beam tightness, while an electronic defocus can have varying amounts of defocusing. In accordance with an exemplary embodiment, a lens and/or convex or concave shaped annular array 1000 can also be provided to aid focusing or defocusing such that any time differential delays can be reduced. Movement of annular array 1000 in one, two or three-dimensions, or along any path, such as through use of probes and/or any conventional robotic arm mechanisms, may be implemented to scan and/or treat a volume or any corresponding space within a region of interest.

Transducer 404 can also be configured in other annular or non-array configurations for imaging/therapy functions. For example, with reference to FIGS. 10C-10F, a transducer can comprise an imaging element 1012 configured with therapy element(s) 1014. Elements 1012 and 1014 can comprise a single-transduction element, e.g., a combined imaging/transducer element, or separate elements, can be electrically isolated 1022 within the same transduction element or between separate imaging and therapy elements, and/or can comprise standoff 1024 or other matching layers, or any combination thereof. For example, with particular reference to FIG. 10F, a transducer can comprise an imaging element 1012 having a surface 1028 configured for focusing, defocusing or planar energy distribution, with therapy elements 1014 including a stepped-configuration lens configured for focusing, defocusing, or planar energy distribution.

In accordance with another aspect of the invention, transducer probe 400 may be configured to provide one, two or three-dimensional treatment applications for focusing acoustic energy to one or more regions of interest. For example, as discussed above, transducer probe 400 can be suitably diced to form a one-dimensional array, e.g., a transducer comprising a single array of sub-transduction elements.

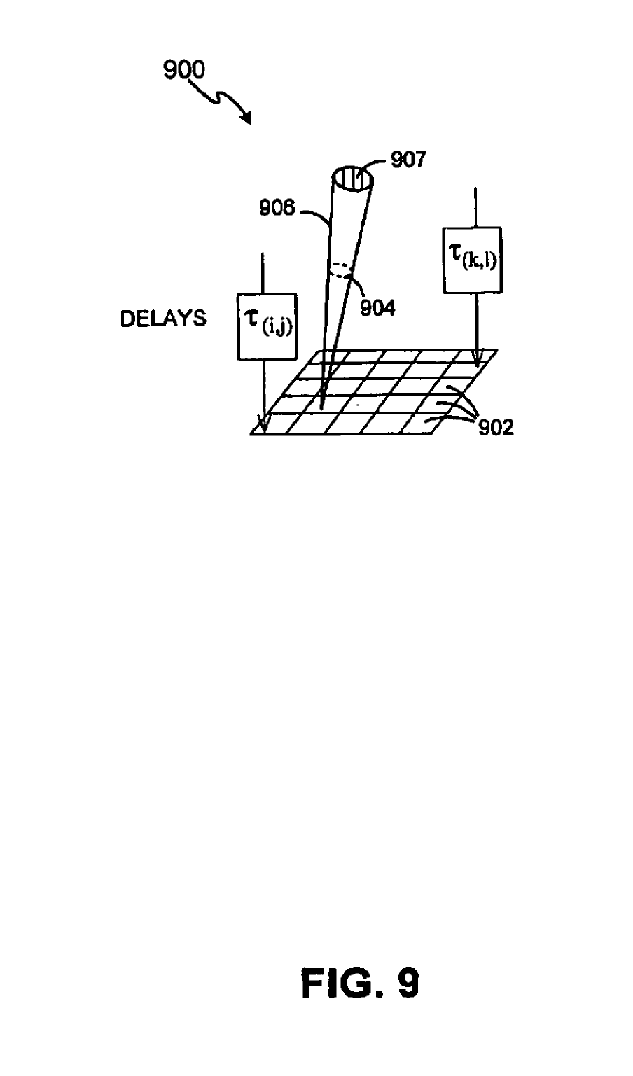

In accordance with another exemplary embodiment, transducer probe 400 may be suitably diced in two-dimensions to form a two-dimensional array. For example, with reference to FIG. 9, an exemplary two-dimensional array 900 can be suitably diced into a plurality of two-dimensional portions 902. Two-dimensional portions 902 can be suitably configured to focus on the treatment region at a certain depth, and thus provide respective slices 904 of the treatment region. As a result, the two-dimensional array 900 can provide a two-dimensional slicing of the image place of a treatment region, thus providing two-dimensional treatment.

In accordance with another exemplary embodiment, transducer probe 400 may be suitably configured to provide three-dimensional treatment. For example, to provide three dimensional treatment of a region of interest, with reference again to FIG. 3, a three-dimensional system can comprise transducer probe 400 configured with an adaptive algorithm, such as, for example, one utilizing three-dimensional graphic software, contained in a control system, such as control system 300. The adaptive algorithm is suitably configured to receive two-dimensional imaging, temperature and/or treatment information relating to the region of interest, process the received information, and then provide corresponding three-dimensional imaging, temperature and/or treatment information.

In accordance with an exemplary embodiment, with reference again to FIG. 9, an exemplary three-dimensional system can comprise a two-dimensional array 900 configured with an adaptive algorithm to suitably receive 904 slices from different image planes of the treatment region, process the received information, and then provide volumetric information 906, e.g., three-dimensional imaging, temperature and/or treatment information. Moreover, after processing the received information with the adaptive algorithm, the two-dimensional array 900 may suitably provide therapeutic heating to the volumetric region 906 as desired.

Alternatively, rather than utilizing an adaptive algorithm, such as three-dimensional software, to provide three-dimensional imaging and/or temperature information, an exemplary three-dimensional system can comprise a single transducer 404 configured within a probe arrangement to operate from various rotational and/or translational positions relative to a target region.

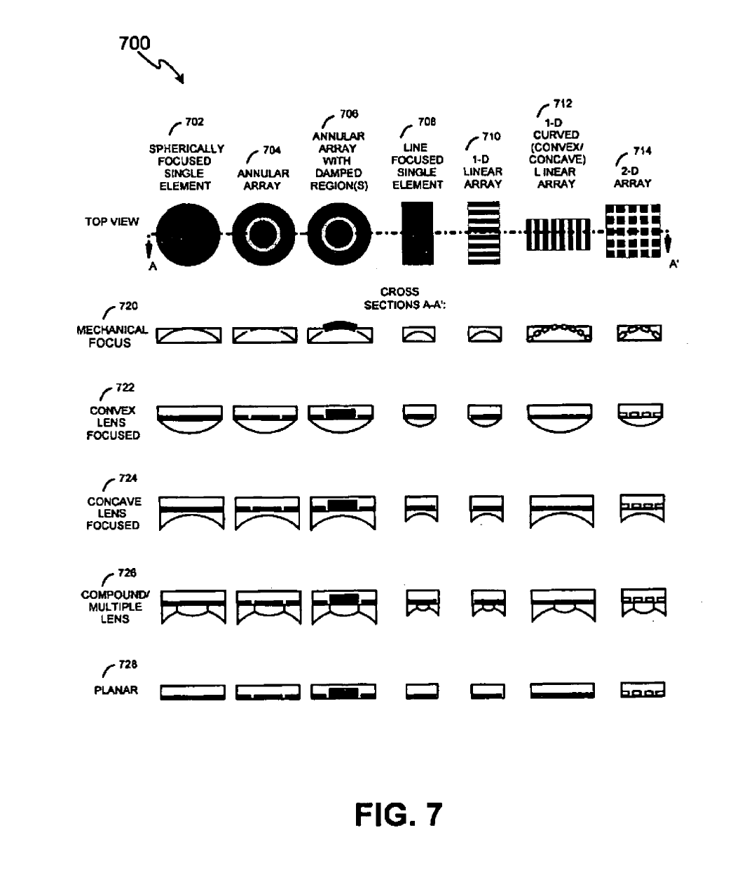

To further illustrate the various structures for transducer 404, with reference to FIG. 7, ultrasound therapy transducer 700 can be configured for a single focus, an array of foci, a locus of foci, a line focus, and/or diffraction patterns. Transducer 700 can also comprise single elements, multiple elements, annular arrays, one-, two-, or three-dimensional arrays, broadband transducers, and/or combinations thereof, with or without lenses, acoustic components, and mechanical and/or electronic focusing. Transducers configured as spherically focused single elements 702, annular arrays 704, annular arrays with damped regions 706, line focused single elements 708, 1-D linear arrays 710, 1-D curvilinear arrays in concave or convex form, with or without elevation focusing, 2-D arrays, and 3-D spatial arrangements of transducers may be used to perform therapy and/or imaging and acoustic monitoring functions. For any transducer configuration, focusing and/or defocusing may be in one plane or two planes via mechanical focus 720, convex lens 722, concave lens 724, compound or multiple lenses 726, planar form 728, or stepped form, such as illustrated in FIG. 10F. Any transducer or combination of transducers may be utilized for treatment. For example, an annular transducer may be used with an outer portion dedicated to therapy and the inner disk dedicated to broadband imaging wherein such imaging transducer and therapy transducer have different acoustic lenses and design, such as illustrated in FIG. 10C-10F.

Various shaped treatment lesions can be produced using the various acoustic lenses and designs in FIGS. 10A-10F. For example, cigar-shaped lesions may be produced from a spherically focused source, and/or planar lesions from a flat source. Concave planar sources and arrays can produce a “V-shaped” or ellipsoidal lesion. Electronic arrays, such as a linear array, can produce defocused, planar, or focused acoustic beams that may be employed to form a wide variety of additional lesion shapes at various depths. An array may be employed alone or in conjunction with one or more planar or focused transducers. Such transducers and arrays in combination produce a very wide range of acoustic fields and their associated benefits. A fixed focus and/or variable focus lens or lenses may be used to further increase treatment flexibility. A convex-shaped lens, with acoustic velocity less than that of superficial tissue, may be utilized, such as a liquid-filled lens, gel-filled or solid gel lens, rubber or composite lens, with adequate power handling capacity; or a concave-shaped, low profile, lens may be utilized and composed of any material or composite with velocity greater than that of tissue. While the structure of transducer source and configuration can facilitate a particular shaped lesion as suggested above, such structures are not limited to those particular shapes as the other spatial parameters, as well as the temporal parameters, can facilitate additional shapes within any transducer structure and source.

Through operation of blood vessel disorder treatment system 100, a method for treatment of blood vessel disorders can be realized that can facilitate effective and efficient therapy without creating chronic injury to human tissue. In one embodiment, the present invention includes a non-invasive method of treatment of vascular tissue at depth using a depth selectable means of energy delivery. In another embodiment, the treatment can be selective, conformable and/or the treatment can cover a whole contiguous surface area. In accordance with various aspects of the present invention, methods to facilitate combining multiple tissue effect mechanisms to achieve a favorable clinical effect are provided.

For example, a user may first select one or more transducer probe configurations for treating a region of interest to achieve a desired effect. The user may select any probe configuration described herein. Because the treatment region ranges from approximately 0 mm to 7 cm, exemplary transducer probes may include, for example, an annular array, a variable depth transducer, a mechanically moveable transducer, a cylindrical-shaped transducer, and the like. As used herein, the term user may include a person, employee, doctor, nurse, and/or technician, utilizing any hardware and/or software of other control systems.

Before, after or during the treatment the region of interest can be imaged by using ultrasound imaging using the same or a separate probe to monitor the treatment region. For example, in one embodiment, the user may image a region of interest in order to plan a treatment protocol. By imaging a region of interest, the user may user the same treatment transducer probe and/or one or more additional transducers to image the region of interest at a high resolution. In one embodiment, the transducer may be configured to facilitate high speed imaging over a large region of interest to enable accurate imaging over a large region of interest.

In another embodiment, ultrasound imaging may include the use of Doppler flow monitoring and/or color flow monitoring. In addition other means of imaging such as photography and other visual optical methods, MRI, X-Ray, PET, infrared or others can be utilized separately or in combination for imaging and feedback of the superficial tissue and the vascular tissue in the region of interest.

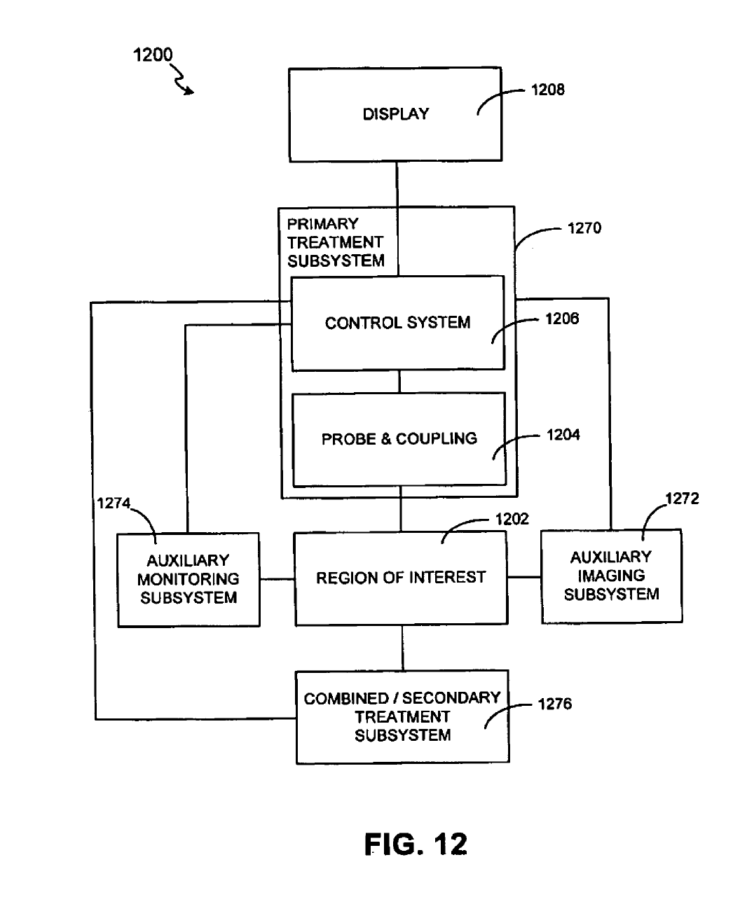

In accordance with another exemplary embodiment, with reference to FIG. 12, an exemplary treatment system 200 can be configured with and/or combined with various auxiliary systems to provide additional functions. For example, an exemplary treatment system 1200 for treating a region of interest 1206 can comprise a control system 1202, a probe 1204, and a display 1208. Treatment system 1200 further comprises an auxiliary imaging modality 1274 and/or auxiliary monitoring modality 1272 may be based upon at least one of photography and other visual optical methods, magnetic resonance imaging (MRI), computed tomography (CT), optical coherence tomography (OCT), electromagnetic, microwave, or radio frequency (RF) methods, positron emission tomography (PET), infrared, ultrasound, acoustic, or any other suitable method of visualization, localization, or monitoring of blood vessels within region-of-interest 1206, including imaging/monitoring enhancements. Such imaging/monitoring enhancement for ultrasound imaging via probe 1204 and control system 1202 could comprise M-mode, persistence, filtering, color, Doppler, and harmonic imaging among others; furthermore an ultrasound treatment system 1270, as a primary source of treatment, may be combined with a secondary source of treatment 1276, including radio frequency (RF), intense pulsed light (IPL), laser, infrared laser, microwave, or any other suitable energy source.

In another exemplary embodiment, an image-treatment method can be used to locate the blood vessel to be treated and then to ablate it non-invasively, while also monitoring the progress of the treatment.

Several embodiments and source conditions can be configured to specifically target the peripheral vascular target pathologies, in a spatially and temporally selective manner. Thus, a treatment protocol is planned by selecting one or more spatial and/or temporal characteristics to provide conformal ultrasound energy to a region of interest. For example, the user may select one or more spatial characteristics to control, including, for example, the use one or more transducers, one or more mechanical and/or electronic focusing mechanisms, one or more transduction elements, one or more placement locations of the transducer relative to the region of interest, one or more feedback systems, one or more mechanical arms, one or more orientations of the transducer, one or more temperatures of treatment, one or more coupling mechanisms and/or the like. In order to facilitate vessel ablation, a transducer that provides for focused ultrasound energy can be used. In order to facilitate ablation of an occlusion, a transducer that provides a lesion similar in shape to that of an occlusion within the vessel, can be used.

In addition, the user may choose one or more temporal characteristics to control in order to facilitate treatment of the region of interest. For example, the user may select and/or vary the treatment time, frequency, power, energy, amplitude and/or the like in order to facilitate temporal control. For more information on selecting and controlling ultrasound spatial and temporal characteristics, see U.S. application Ser. No. 11/163,148, entitled “Method and System for Controlled Thermal Injury,” filed Oct. 6, 2005 and previously incorporated herein by reference.

After planning of a treatment protocol is complete, the treatment protocol can be implemented. That is, a transducer system can be used to deliver ultrasound energy to a treatment region to ablate select tissue in order to facilitate blood vessel disorder treatment. By delivering energy, the transducer may be driven at a select frequency, a phased array may be driven with certain temporal and/or spatial distributions, a transducer may be configured with one or more transduction elements to provide focused, defocused and/or planar energy, and/or the transducer may be configured and/or driven in any other ways hereinafter devised.

In one exemplary embodiment, in order to treat particular peripheral vascular deformities that require treatment in particular anatomical sites (for example, the lower limb region), an ultrasound transducer is taken and coupled to the skin tissue using one of the numerous coupling media, such as water, mineral oils, gels, etc. This transducer can be configured geometrically and/or electronically to selectively deposit energy at a particular depth below the skin surface. Alternatively, the spatial deposition of energy may be planned to be deposited in a defined pattern based on the imaging of the region of interest before commencing therapy.

In one exemplary embodiment, ultrasound energy is delivered or deposited at a selective depth to facilitate ablation of a vessel. The ultrasound energy deposition is preferably selectable but not limited to surface of skin tissue ranging from 0.1 to 5 mm in diameter at a depth of up to 7 mm. The power used to deliver the ultrasound source at one location may range from, for example, about 5 W to about 50 W, and a corresponding source frequency may range from about 2 MHz to about 5 MHz.

In another exemplary embodiment, ultrasound energy is delivered at a selective depth to facilitate ablation of an occlusion within a vessel. The ultrasound energy deposition is preferably selectable but not limited to surface of skin tissue ranging from 3 to 20 mm in diameter at a depth of up to 70 mm. The power used to deliver the ultrasound source at one location may range from, for example, about 5 W to about 200 W, and a corresponding source frequency may range from about 2 MHz to about 20 MHz. If treatment of the occlusion does not increase blood flow through the region of interest the exemplary transducer system can be used to further ablate the occlusion.

In another exemplary embodiment, the ultrasound energy can also be combined with one or more number of pharmaceutical formulations that are currently prescribed for the treatment of peripheral vascular disorders such as sclerosing agents for varicose and spider veins, and energy activated drugs for port wine stains and hemangiomas. The ultrasound energy and/or formulations may acts synergistically by causing one or more effects to a region of interest. For example, the ultrasound energy may, (1) increasing activity of the agents due to the thermal and non-thermal mechanisms, (2) reduced requirement of overall drug dosage, as well as reducing the drug toxicity, (3) increase local effect of drug in a site selective manner. In yet another exemplary embodiment, treatment of blood vessel disorders can be achieved by combining at least two of ablation, cavitation, and streaming.

Once the treatment protocol has been implemented, the region of tissue may have one or more biological responses in reaction to the treatment. For example, in one embodiment, the vessel responds by increased blood flow as an occlusion within the vessel becomes unobstructed. In another embodiment, the vessel responds to ablation by disintegrating within the body.

Upon treatment, the steps outlined above can be repeated one or more additional times to provide for optimal treatment results. Different ablation sizes and shapes may affect the recovery time and time between treatments. For example, in general, the larger the surface area of the treatment lesion, the faster the recovery. The series of treatments can also enable the user to tailor additional treatments in response to a patient's responses to the ultrasound treatment.

The present invention has been described above with reference to various exemplary embodiments. However, those skilled in the art will recognize that changes and modifications may be made to the exemplary embodiments without departing from the scope of the present invention. For example, the various operational steps, as well as the components for carrying out the operational steps, may be implemented in alternate ways depending upon the particular application or in consideration of any number of cost functions associated with the operation of the system, e.g., various steps may be deleted, modified, or combined with other steps. These and other changes or modifications are intended to be included within the scope of the present invention, as set forth in the following claims.

What is claimed is:

1. A non-invasive method for treating a spider vein, the method comprising:
positioning an ultrasound probe on a skin surface, the ultrasound probe comprising at least one ultrasound therapy element;
imaging a region of interest under the skin surface, wherein the region of interest comprises a spider vein;
using the at least one ultrasound therapy element to create a plurality of thermal lesions in at least a portion of the spider vein in the region of interest,
wherein the at least one ultrasound therapy element is configured to deliver ultrasound energy at a depth of up to about 7 mm below the skin surface.

2. The method of claim 1,
wherein the at least one ultrasound therapy element delivers energy at a frequency in a range of about 2 MHz to about 20 MHz,
wherein the step of imaging the region of interest comprises ultrasound imaging, and
further comprising moving the at least one ultrasound therapy element along a linear path using an automated motion mechanism.

3. The method of claim 1, wherein the using the at least one ultrasound therapy element comprises adjustable control of spatial parameters and temporal parameters of the ultrasound probe to generate thermal lesions of specifically targeted shapes, sizes or orientations in at least a portion of the region of interest.

4. The method of claim 1, wherein the using the at least one ultrasound therapy element comprises using a motion mechanism coupled to the at least one ultrasound therapy element within the ultrasound probe.

5. The method of claim 1, wherein the using the at least one ultrasound therapy element comprises using electronic phase focusing of the at least one ultrasound therapy element to position a focus for delivery of the energy to create a thermal lesion in the spider vein.

6. The method of claim 1, wherein the step of creating the plurality of thermal lesions comprises producing a discrete locus of spaced conformal lesions based on adjustable control of spatial parameters and temporal parameters.

7. The method of claim 1, wherein the step of creating the plurality of thermal lesions comprises producing a one-, two- or three-dimensional matrix of spaced lesions in the spider vein.

8. The method of claim 1, further comprising monitoring the spider vein for further planning, assessing of results, or providing feedback.

9. The method of claim 1, wherein the ultrasound energy is delivered at a frequency in a range of about 2 MHz to about 20 MHz.

10. A method for providing treatment of a vascular disorder, the method comprising:
targeting therapeutic acoustic waves, via at least one ultrasound treatment element housed within an ultrasound probe, through a skin surface to treat a blood vessel under the skin surface; and
activating phase focusing of the at least one ultrasound treatment element for controllably creating a plurality of thermal lesions along a line at a depth up to about 7 mm below the skin surface,
wherein the activating phase focusing comprises varying at least one electronic time delay controlled by a control system in communication with the ultrasound probe,
wherein the plurality of thermal lesions treats a vascular disorder in the blood vessel, and
wherein the vascular disorder is selected from the group consisting of a spider vein, a hemangioma and a port wine stain.

11. The method of claim 10, wherein the ultrasound energy is delivered at a frequency in a range of about 2 MHz to about 20 MHz.

12. The method of claim 10, further comprising imaging a region below the skin surface.

13. The method of claim 10, further comprising moving the treatment element along the line using an automated motion mechanism.

14. The method of claim 10, wherein the plurality of thermal lesions comprise a matrix of spaced treatment spots that are spaced using an automated motion mechanism.

15. A method for treating a vascular disorder, the method comprising:
positioning an ultrasound probe on a skin surface proximate to at least one aberrant blood vessel;
wherein the ultrasound probe comprises an ultrasound therapy element configured to deliver ultrasound energy at a frequency in a range of about 2 MHz to about 20 MHz;
selecting an ultrasound probe configuration based on at least one of a spatial parameter and a temporal parameter;
verifying the at least one of a spatial parameter and a temporal parameter of the probe; and
using a motion mechanism to move the ultrasound therapy element to create a plurality of thermal lesions in at least a portion of the aberrant blood vessel; and
using an ultrasound imaging element to image a region of interest under the skin surface comprising the aberrant blood vessel.

16. The method of claim 15, wherein the imaging element is configured for at least one of Doppler flow monitoring and color flow monitoring.

17. The method of claim 15, wherein the using the motion mechanism to move the ultrasound therapy element comprises controlling spatial parameters and temporal parameters of the probe to generate conformal lesions of specifically targeted shapes, sizes and orientations.

18. The method of claim 15, wherein the using the motion mechanism comprises producing a matrix of spaced treatment spots comprising at least one of a two-dimensional and three-dimensional matrix of lesions in the region of interest along a scanned pattern created by scanning of the probe, wherein the producing the matrix of spaced treatment spots comprises producing a discrete locus of spaced lesions based on adjustable control of spatial parameters and temporal parameters.

19. The method of claim 15, further comprising treating the blood vessel with any one in the group consisting of cavitational, hydrodynamic, and resonance induced tissue effects.

20. The method of claim 15, wherein the plurality of thermal lesions comprise a matrix of spaced treatment spots that are spaced using the motion mechanism.
